(19) United States
(12) Patent Application Publication
(10) Pub. No.: US 2008/0312515 A1
(43) Pub. Date: Dec. 18, 2008

(54) System and method for providing cardiovascular disorder diagnosis services

(75) Inventors: Chan-Hyun Youn, Daejeon (KR); Chang-Hee Han, Daejeon (KR); Youngjoo Han, Daejeon (KR); Byung-Jin Kim, Daejeon (KR); Jin-Ho Kim, Daejeon (KR); Eun Bo Shim, Chuncheon-si (KR)

Correspondence Address: BACON & THOMAS, PLLC, 625 SLATERS LANE, FOURTH FLOOR, ALEXANDRIA, VA, 22314-1176, US

(73) Assignee: RESEARCH AND INDUSTRIAL COOPERATION GROUP, Daejeon (KR)

(21) Appl. No.: 12/000,038

(22) Filed: Dec. 7, 2007

(30) Foreign Application Priority Data
Jun. 18, 2007 (KR) 10-2007-0059462

(51) Int. Cl. A61B 5/00

(52) U.S. Cl. 600/300; 705/2

(57) ABSTRACT

System and method provide an on-line high-performance diagnosis service for cardiovascular disorders. A client requests a high-performance diagnosis by transmitting real electrocardiographic treatment data and magnetocardiographic treatment data of a human body being a treatment object and virtual heart simulation parameters to a medical service server. The medical service server, in response to the diagnosis request, analyzes the real electrocardiographic treatment data to generate an electrocardiographic analysis result, and performs a virtual heart simulation using the simulation parameters to generate a pseudo electrocardiogram and magnetocardiogram. Further, the medical service server determines a disease state of the human body on the basis of the electrocardiographic analysis result, the magnetocardiographic treatment data and the pseudo electrocardiogram and magnetocardiogram, and generates definitive diagnosis data through comparison among the real magnetocardiographic treatment data, the electrocardiographic analysis result, the disease state, and a diagnosis criteria. The definitive diagnosis data is provided to the client.

### FIELD OF THE INVENTION

The present invention relates to a cardiovascular disorder diagnosis service and, more particularly, to a method and system for on-line high-performance diagnosis of cardiovascular disorders using real electrocardiographic and/or magnetocardiographic treatment data of human bodies.

### BACKGROUND OF THE INVENTION

As well known in the art, cardiovascular disorders, such as myocardial infarction, angina pectoris, cardiac failure, arteriosclerosis, embolism, hypertension, atherosclerosis and thrombus, prevail throughout highly developed countries. In particular, cardiovascular disorders, cancer, and cerebrovascular diseases are leading causes of death.

Electrocardiography has been used to diagnose cardiovascular disorders, and has an advantage of portability and cost. Since electrocardiography has a limit of diagnosis accuracy, active researches has been conducted to raise the accuracy of cardiovascular disorder diagnosis through, for example, the increased number of channels and long-term data analysis. Complexity in signal processing increases accordingly therewith, and there still exists a limit of sensitivity to cardiovascular disorders and of confidence in made assumptions.

To solve above problems, magnetocardiography having a diagnostic accuracy higher than that of electrocardiography is applied to cardiovascular disorder diagnosis. Magnetocardiography has also some limitations. For example, the magnetocardiography has a limit in exact diagnosis of disease symptoms in which abnormalities of the heart can be detected, but can not diagnosis what disease is related to the abnormalities or on what region of the heart shows the abnormalities.

On the other hand, real electrocardiographic and magnetocardiographic waveforms can be compared with those generated by a simulation. A virtual heart is a technique to diagnose diseases on the basis of electrophysiological properties initially input to the simulation and the degree of agreement between the real and generated waveforms. Hence, it is necessary to complement individual diagnosis techniques each other for a high-performance integrated diagnosis system.

In connection with e-Health systems measuring the cardiovascular system, patient state sensing, integration with mobile appliances such as personal digital assistants (PDA), and integration with Grid technology has been major research topics. That is, existing e-Health systems have failed to consider integrated diagnosis. Management and integration of physically distributed vast amount of data, which is essential to an e-Health system for cardiovascular disorder diagnosis, have not been fully studied.

Accordingly, it is necessary to develop a new diagnosis technique enabling both integration of existing diagnosis techniques and integrated management of distributed treatment data.

### SUMMARY OF THE INVENTION

Therefore, an object of the present invention is to provide a method and system for providing cardiovascular disorder diagnosis services, wherein high-performance diagnosis services are delivered on-line via a network by way of integrated cardiovascular disorder diagnoses.

Another object of the present invention is to provide a method and system for providing cardiovascular disorder diagnosis services, wherein high-performance diagnosis services are delivered on-line on the basis of a real electrocardiogram and magnetocardiogram obtained from a human body and a pseudo electrocardiogram and magnetocardiogram obtained through a virtual heart simulation.

Still another object of the present invention is to provide a method and system for providing cardiovascular disorder diagnosis services, wherein efficient resource management in on-line diagnosis services is achieved through integrated management of definitive diagnosis data on cardiovascular disorders that is stored in a plurality of distributed data repositories.

In accordance with an aspect of the present invention, there is provided a diagnosis system for providing cardiovascular disorder diagnosis services through a network, including:

a client group having one or more clients, each of which transmits real electrocardiographic treatment data and magnetocardiographic treatment data of a human body being a treatment object along with a cardiovascular disorder diagnosis request through the network, receives definitive diagnosis data as a reply to the cardiovascular disorder diagnosis request through the network; and

a medical service server for analyzing the real electrocardiographic treatment data received through the network from the client in accordance with a task schedule utilizing available resource information, determining a disease state of the human body on the basis of the electrocardiographic analysis result, the real magnetocardiographic treatment data, and pseudo electrocardiogram and magnetocardiogram obtained through a virtual heart simulation, creating definitive diagnosis data on cardiovascular disorders of the human body on the basis of the real magnetocardiographic treatment data, the electrocardiographic analysis result and the determined disease state, and transmitting the created definitive diagnosis data through the network to the client.

In accordance with another aspect of the present invention, there is provided a method of providing cardiovascular disorder diagnosis services through a network, including:

requesting, by a client, a high-performance diagnosis on cardiovascular disorders by transmitting real electrocardiographic treatment data and magnetocardiographic treatment data of a human body being a treatment object and virtual heart simulation parameters through the network to a medical service server;

analyzing, by the medical service server, in response to the high-performance diagnosis request, the real electrocardiographic treatment data to generate an electrocardiographic analysis result;

performing, by the medical service server, a virtual heart simulation using the simulation parameters to generate a pseudo electrocardiogram and magnetocardiogram;

determining, by the medical service server, a disease state of the human body on the basis of the electrocardiographic analysis result, the magnetocardiographic treatment data, and the pseudo electrocardiogram and magnetocardiogram;

generating, by the medical service server, definitive diagnosis data for cardiovascular disorders through comparison between the real magnetocardiographic treatment data, the electrocardiographic analysis result, the disease state, and a diagnosis criteria; and

transmitting, by the medical service server, the definitive diagnosis data through the network to the client.

In accordance with further another aspect of the present invention, there is provided method of providing cardiovascular disorder diagnosis services through a network, including:

requesting, by a client, a high-performance diagnosis on cardiovascular disorders by transmitting real electrocardiographic treatment data and magnetocardiographic treatment data of a human body being a treatment object and virtual heart simulation parameters through the network to a medical service server;

performing, by medical service server, in response to the high-performance diagnosis request, an analysis on the real electrocardiographic treatment data in a distributed manner to generate an electrocardiographic analysis result, and detecting whether or not there is an abnormality associated with ischemic heart diseases on the basis of the electrocardiographic analysis result and diagnosis criteria from a diagnosis reference table;

detecting, by medical service server, if the abnormality associated with the ischemic heart diseases is not detected, whether or not there is an abnormality associated with tachycardia or bradycardia on the basis of the diagnosis criteria from the diagnosis reference table;

creating, by medical service server, if the abnormality associated with tachycardia or bradycardia is not detected, definitive diagnosis data indicating a normal state of the human body, and sending the definitive diagnosis data through the network to the client;

detecting, by medical service server, if the abnormality associated with tachycardia or bradycardia is detected, whether or not there is an abnormality associated with ischemic heart diseases on the basis of the real magnetocardiographic treatment data and the diagnosis criteria from the diagnosis reference table;

creating, by medical service server, if the abnormality associated with ischemic heart diseases is not detected, definitive diagnosis data containing an indication of tachycardia or bradycardia in the human body, and sending the definitive diagnosis data through the network to the client;

deriving, by medical service server, if an abnormality associated with ischemic heart diseases is detected on the basis of the real electrocardiographic and/or magnetocardiographic treatment data, a pseudo electrocardiogram and magnetocardiogram through a distributed virtual heart simulation with the simulation parameters;

determining, by medical service server, a disease state of cardiovascular disorders of the human body on the basis of the electrocardiographic analysis result, the real magnetocardiographic treatment data, and the pseudo electrocardiogram and magnetocardiogram; and

creating, by medical service server, definitive diagnosis data through comparison among the real magnetocardiographic treatment data, the electrocardiographic analysis result, disease state and the diagnosis criteria, and sending the definitive diagnosis data through the network to the client.

### BRIEF DESCRIPTION OF THE DRAWINGS

The above and other objects and features of the present invention will become apparent from the following description of embodiments given in conjunction with the accompanying drawings, in which:

FIG. 1 is a schematic view illustrating a cardiovascular disorder diagnosis system in accordance with an embodiment of the present invention;

FIG. 2 is a detail block diagram of a client in FIG. 1;

FIG. 3 is a detail block diagram of a medical service server in FIG. 1;

FIG. 4 is a detail block diagram of an information storage/management module in FIG. 3;

FIG. 5 is a detail block diagram of an electrocardiographic analysis module in FIG. 3;

FIG. 6 is a detail block diagram of a virtual heart simulation module in FIG. 3;

FIG. 7 is a detail block diagram of a cardiovascular disorder diagnosis module in FIG. 3;

FIG. 8 is a block diagram illustrating a distributed-data processing module in FIG. 3;

FIGS. 9 and 10 are flow charts illustrating a procedure of providing a high-performance diagnosis service for cardiovascular disorders to clients in accordance with another embodiment of the present invention;

FIG. 11 is a flow chart illustrating a procedure of providing a client with cardiovascular disorder diagnosis data to achieve an integrated management service;

FIG. 12 is a graph showing a pseudo electrocardiogram generated by a virtual heart simulation;

FIG. 13 is a graph showing a pseudo magnetocardiogram generated by a virtual heart simulation;

FIG. 14 is a graph showing a pseudo magnetocardiographic angle waveform generated by a virtual heart simulation; and

FIG. 15 is a flow chart illustrating a procedure of providing a diagnosis service for tachycardia, bradycardia and ischemic heart diseases through selective performance of an electrocardiographic analysis and virtual heart simulation.

### DETAILED DESCRIPTION OF THE EMBODIMENTS

Hereinafter, embodiments of the present invention will be described in detail with reference to the accompanying drawings.

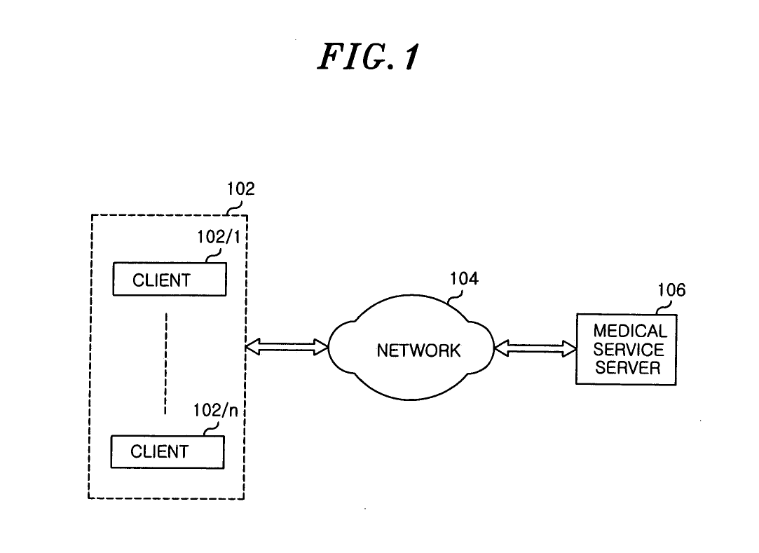

Referring now to FIG. 1, there is shown a schematic view illustrating a cardiovascular disorder diagnosis system according to the present invention.

As shown in FIG. 1, the cardiovascular disorder diagnosis system includes a client group 102 composed of a plurality of clients 102/1 to 102/n, a network 104 such as an Internet-based network, and a medical service server 106.

The clients 102/1 to 102/n in the client group 102 may be, for example, individual server systems or personal computers installed at hospitals or clinics. Each of the clients 102/1 to 102/n, in response to an operation of a user (for example, a doctor), transmit treatment data, that is obtained through medical instruments for cardiovascular disorder diagnoses, (the data being related to a real electrocardiogram, magnetocardiogram of a human body being a treatment object, virtual heart simulation parameters and the like) through the network 104 to the medical service server 106 along with a service request of a high-performance diagnosis on cardiovascular disorders of a patient. The client then can receive a definitive diagnosis result from the medical service server 106.

When a client receives a request for definitive diagnosis data stored in its own data storage block from the other client through the medical service server 106, the client retrieves the requested definitive diagnosis data from its data storage block, and sends the definitive diagnosis data to the other client through the medical service server 106. In this case, the client acts as a data repository for the other client.

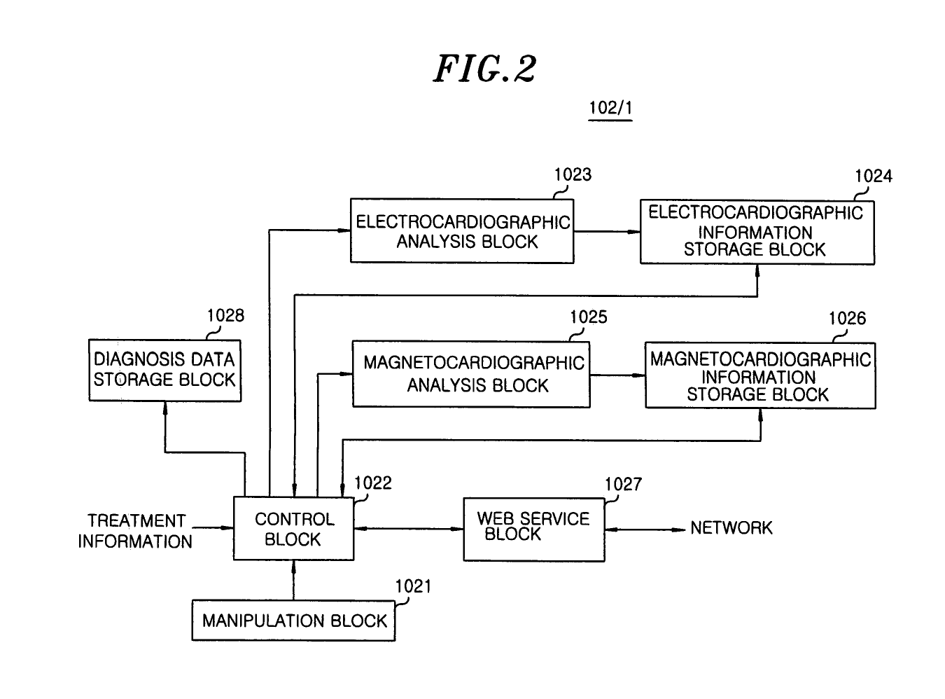

FIG. 2 is a detail block diagram illustrating a client in FIG. 1.

As shown in FIG. 2, the client includes a manipulation block 1021, a control block 1022, an electrocardiographic analysis block 1023, an electrocardiographic information storage block 1024, a magnetocardiographic analysis block 1025, a magnetocardiographic information storage block 1026, an Web service block 1027, and a diagnosis data storage block 1028.

The manipulation block 1021 is manipulation means (for example, a keypad, a mouse and a touch panel) for controlling the overall operation of the clients, and sends various manipulation signals (e.g., command signals, virtual heart simulation parameters and the like) generated by actions of the user to the control block 1022.

The control block 1022 may include a microprocessor for controlling the overall operation of the client. The control block 1022 receives treatment information such as real electrocardiographic and magnetocardiographic information on a human body) from a medical instrument or computer (not shown), and forwards the treatment information to the electrocardiographic analysis block 1023 and magnetocardiographic analysis block 1025.

The electrocardiographic analysis block 1023 analyzes electrocardiographic signals of probable diseases (for example, tachycardia, bradycardia and ischemic heart disease) using an electrocardiographic analysis algorithm, and stores the analysis result in the electrocardiographic information storage block 1024 as real electrocardiographic treatment data of a human body.

Similarly, the magnetocardiographic analysis block 1025 analyzes magnetocardiographic signals of the probable diseases using a magnetocardiographic analysis algorithm, and stores the analysis result in the magnetocardiographic information storage block 1026 as real magnetocardiographic treatment data of a human body.

Hence, the user can diagnose cardiovascular disorders of a human body on the basis of analysis results obtained by the electrocardiographic analysis block 1023 and magnetocardiographic analysis block 1025 using real electrocardiographic and magnetocardiographic information. These local analysis results are merely a fast-track analysis result rather than a high-performance analysis result requiring relatively high computing power.

The user can extract the real electrocardiographic and magnetocardiographic treatment data of a human body from the electrocardiographic information storage block 1024 and magnetocardiographic information storage block 1026, and send the extracted real electrocardiographic and magnetocardiographic treatment data along with the virtual heart simulation parameters in order to request for a high-performance diagnosis on cardiovascular disorders via the network 104 to the medical service server 106. Access to the medical service server 106 is made through user access control, i.e., log-in) and service usage level control.

More specifically, in response to a service request for a high-performance diagnosis from the user, the control block 1022 obtains user authentication, and sends the virtual heart simulation parameters from the manipulation block 1021 and the real electrocardiographic and magnetocardiographic treatment data through the Web service block 1027 and the network 104, to the medical service server 106, in order for a high-performance cardiovascular disorder diagnosis.

The Web service block 1027 includes a Web browser for Web access. The Web service block 1027 converts the real electrocardiographic and magnetocardiographic treatment data and the virtual heart simulation parameters from the control block 1022 into a Web Services Description Language (WSDL) description and sends the WSDL description through the network 104. Further, the Web service block 1027 receives a WSDL description indicative of the definitive cardiovascular disorder diagnosis result through the network 104, restores the original data restored from the WSDL description, and sends the original data to the control block 1022.

The control block 1022 receives the definitive diagnosis data, in response to the diagnosis service request, from the medical service server 106, and stores the definitive diagnosis data in the diagnosis data storage block 1028. Additionally, the control block 1022 extracts, in response to a request for definitive diagnosis data from the other client, the requested definitive diagnosis data from the diagnosis data storage block 1028, and sends the definitive diagnosis data to the medical service server 106. That is, any client can receive and refer to the definitive diagnosis data on cardiovascular disorders stored in the other client. That is, any client may act as a data repository for the other client. To do it, the diagnosis data storage block 1028 stores various definitive diagnosis data on cardiovascular disorders received from the medical service server 106 as a reply to high-performance diagnosis requests.

Although, in FIG. 2, the electrocardiographic information storage block 1024, magnetocardiographic information storage block 1026 and diagnosis data storage block 1028 are illustrated as separate components, the present invention is not limited thereto. These components may also be implemented with an integrated single data storage, and each component may correspond to a separately allocated storage space in the single data storage.

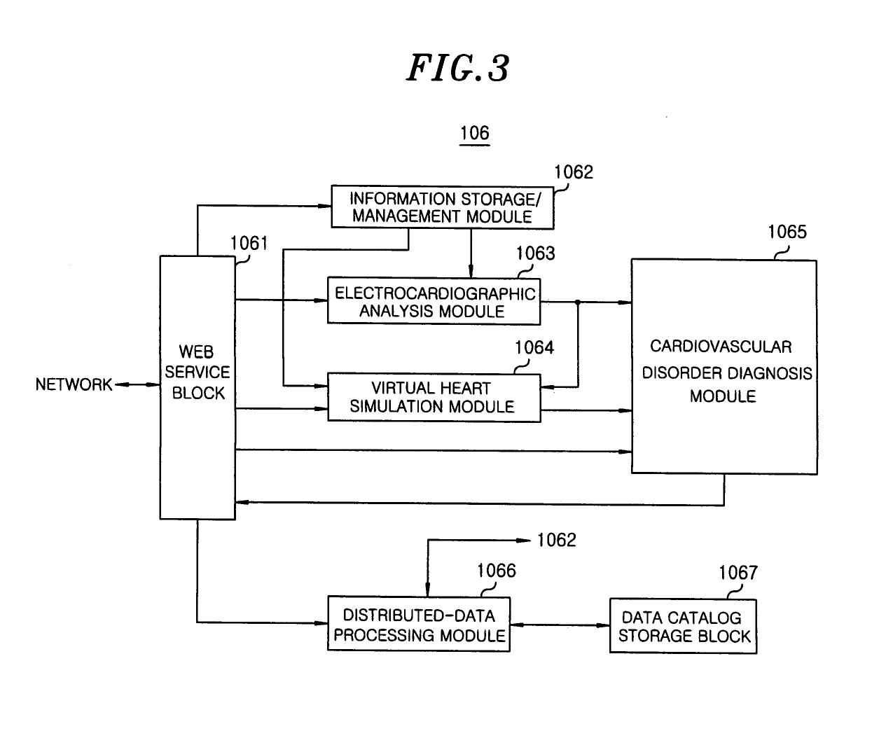

Referring back to FIG. 1, the medical service server 106 analyzes the real electrocardiographic treatment data along with the diagnosis service request, which has been received through the network 104 from the client, to generate an electrocardiographic analysis result using a high-performance electrocardiographic analysis algorithm utilizing available resource information; performs a virtual heart simulation using received parameters to derive a pseudo electrocardiogram and magnetocardiogram; and performs an analysis of agreement between the electrocardiographic analysis result and real magnetocardiographic treatment data and the pseudo electrocardiogram and magnetocardiogram. The medical service server 106 then determines the disease state of cardiovascular disorders with reference to the degree of agreement; and performs definitive cardiovascular disorder diagnosis of the human body on the basis of comparison between the determined disease state, the electrocardiographic analysis result, real magnetocardiographic treatment data, and diagnosis criteria for cardiovascular disorders. The definitive diagnosis data of the human body through the network 104 provided to the requesting client. Various functions of the medical service server 106 are described further in connection with FIGS. 3 to 8.

FIG. 3 is a detail block diagram of the medical service server 106 in FIG. 1.

As shown in FIG. 3, the medical service server 106 includes a Web service block 1061, information storage/management module 1062, electrocardiographic analysis module 1063, virtual heart simulation module 1064, cardiovascular disorder diagnosis module 1065, distributed-data processing module 1066, and data catalog storage block 1067.

The Web service block 1061 in FIG. 3 is substantially identical in function to the Web service block 1027 in FIG. 2. The Web service block 1061 receives the WSDL description data (for example, the user access control information, the real electrocardiographic and magnetocardiographic treatment data, and the virtual heart simulation parameters) through the network 104, restores the original data restored from the WSDL description data, and selectively forwards the original data to the information storage/management module 1062, electrocardiographic analysis module 1063, the virtual heart simulation module 1064, the cardiovascular disorder diagnosis module 1065, and the distributed-data processing module 1066. The Web service block 1061 converts the definitive cardiovascular disorder diagnosis data from the cardiovascular disorder diagnosis module 1065 and the distributed-data processing module 1066 into WSDL description data, and sends the WSDL description data through the network 104.

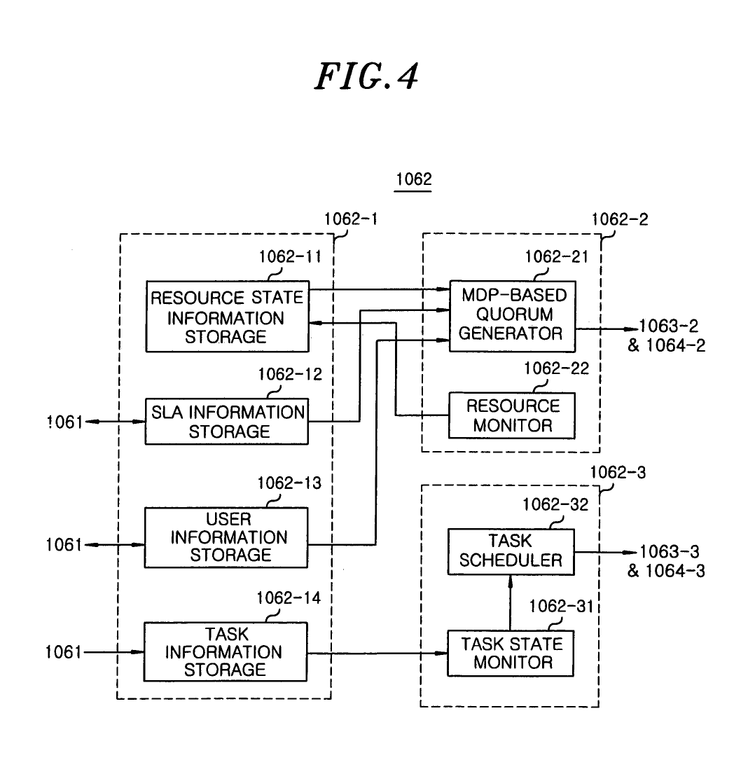

The information storage/management module 1062 manages user personal information (for example, names, birth dates, jobs, home/office addresses, home/office phone numbers, e-mail addresses, and cellular phone numbers), user class (service access level) information, and user access control information based on service access levels. Further, the information storage/management module 1062 performs resource management related to, for example, system quality factors, network quality factors and the like; a task schedule management; and an user task history management related to, for example, the number of logins per user, performed tasks per user and the like. These operations are further described in connection with FIG. 4.

FIG. 4 is a detail block diagram of the information storage/management module 1062 in FIG. 3.

As shown in FIG. 4, the information storage/management module 1062 includes an information storage module 1062-1, resource management module 1062-2, and task management module 1062-3. The information storage module 1062-1 includes a resource state information storage 1062-11, service level agreement (SLA) information storage 1062-12, user information storage 1062-13, and task information storage 1062-14. The resource management module 1062-2 includes a Markov decision process (MDP)-based quorum generation module 1062-21 and resource monitoring block 1062-22. The task management module 1062-3 includes a task state monitoring block 1062-31 and task scheduler 1062-32.

The resource state information storage 1062-11 stores resource state information (e.g., CPU usage, memory usage, etc, and network state information (e.g., bandwidths, latencies, jitters, etc) using resource monitoring information from the resource monitoring block 1062-22. The resource state information is provided to the MDP-based quorum generation module 1062-21.

The SLA information storage 1062-12 stores resource quality information necessary for SLA pursuant to a service level (class) of each user. Resource quality factors may include system quality factors related to, for example, the CPU, memory and storage, and network quality factors such as bandwidths, latencies and loss rates. The resource quality information is provided to the MDP-based quorum generation module 1062-21.

The user formation storage 1062-13 stores therein personal information, task history information, and service level information for user management. The user information storage 1062-13 performs user access control (i.e., authentication of a user having valid usage rights) on the basis of user class information, and provides the task history information to the MDP-based quorum generation module 1062-21.

The task information storage 1062-14 stores task state information (for example, a currently requested task, currently running task and previously executed task) received through the Web service block 1061 from each client, and provides the task state information to the task state monitoring block 1062-31.

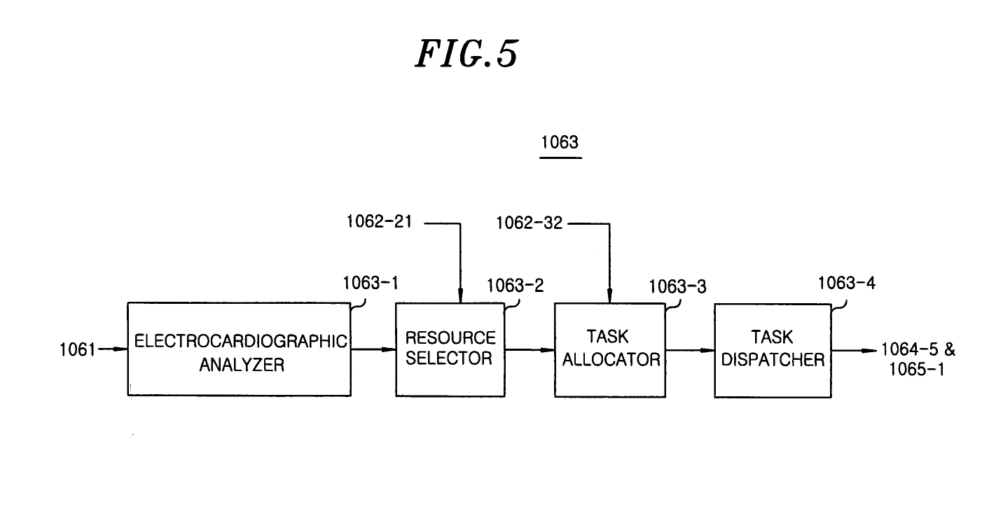

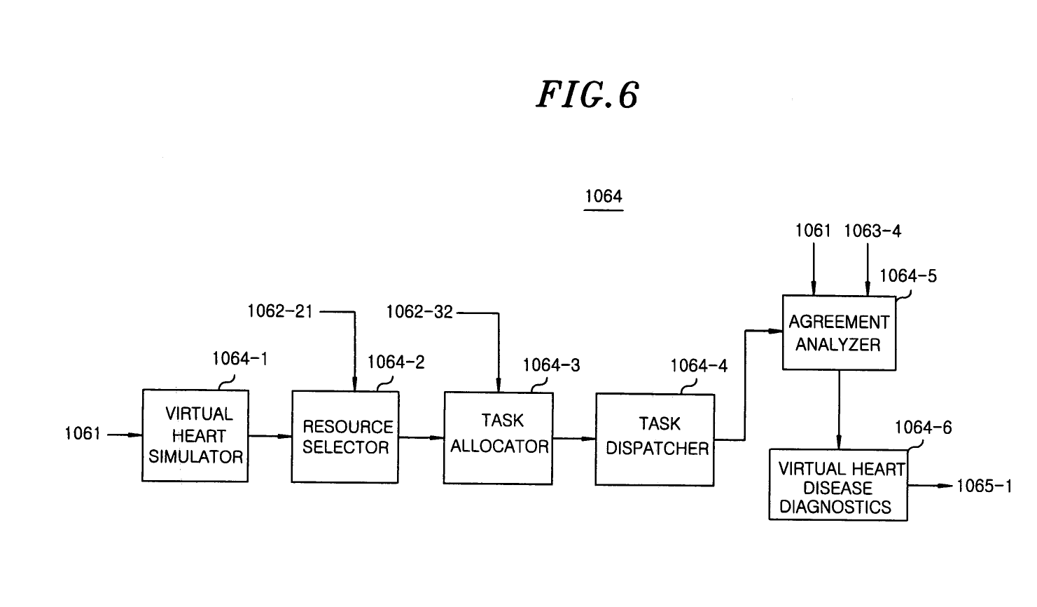

In the resource management module 1062-2, the MDP-based quorum generator 1062-21 creates optimum available resource information (for example, a list of resources available upon processing demand from a user, and states of the available resources) using various information (for example, CPU usage, memory usage, network states, system quality factors, network quality factors, and task histories) from the 11resource state information storage 1062-11, SLA information storage 1062-12, and user information storage 1062-13. The created optimum available resource information is provided to a resource selection block 1063-2 (FIG. 5) of the electrocardiographic analysis module 1063 and to a resource selection block 1064-2 (FIG. 6) of the virtual heart simulation module 1064.

The resource monitoring block 1062-22 monitors the states of actually available resources (for example, CPU usage, memory usage, network states related to bandwidths, latencies and jitters), creates resource monitoring information, and provides the created resource monitoring information to the 11resource state information storage 1062-11.

In the task management module 1062-3, the task state monitoring block 1062-31 receives the task state information from the task information storage 1062-14, and provides the task state information to the task scheduler 1062-32.

The task scheduler 1062-32 creates task scheduling information (for example, a list of currently requested tasks and states of currently running tasks including start times and planned completion times) using the task stat information from the task state monitoring block 1062-31. The task scheduler 1062-32 provides the created task scheduling information to a task allocator 1063-3 (FIG. 5) of the electrocardiographic analysis module 1063 and to a task assignment block 1064-3 (FIG. 6) of the virtual heart simulation module 1064.

Referring back to FIG. 3, the electrocardiographic analysis module 1063 analyzes real electrocardiographic treatment data of a human body (Grid-based electrocardiographic analysis) from the Web service block 1061 on the basis of information regarding a user-requested task, user service level, available computing resource and task schedule, to thereby produce the electrocardiographic analysis result. The electrocardiographic analysis result is then provided to the virtual heart simulation module 1064 and cardiovascular disorder diagnosis module 1065. These functions are described further in connection with FIG. 5.

FIG. 5 is a detail block diagram of the electrocardiographic analysis module 1063 in FIG. 3.

As shown in FIG. 5, the electrocardiographic analysis module 1063 includes an electrocardiographic analyzer 1063-1, resource selector 1063-2, task allocator 1063-3, and task dispatcher 1063-4.

The electrocardiographic analyzer 1063-1 receives user requested task information (for example, a disease name such as tachycardia, bradycardia, ischemic heart disease, or the like) and user service level from the Web service block 1061, and provides the received data to the resource selector 1063-2.

The resource selector 1063-2 chooses resources to be used for task processing (for example, computing resources such as a cluster or desktop) on the basis of user requested task information from the electrocardiographic analyzer 1063-1 and optimum available resource information from the MDP-based quorum generation module 1062-21 in FIG. 4. Information regarding the resources to be used is transferred to the task allocator 1063-3.

The task allocator 1063-3 selects resources to be allocated to the task on the basis of the scheduling information from the task scheduler 1062-32 in FIG. 4 with respect to the task information and the resource assignment information from the resource selector 1063-2. The resource-to-task assignment information is transferred to the task dispatcher 1063-4.

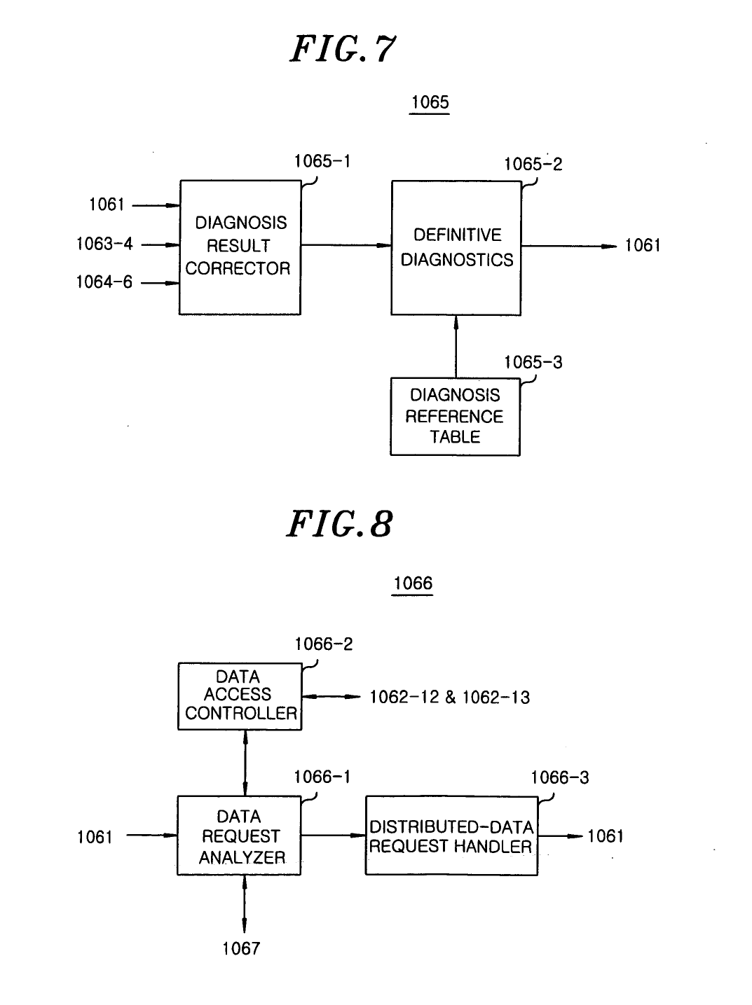

The task dispatcher 1063-4 processes an electrocardiographic analysis task in a distributed manner on the basis of, for example, a Grid middleware-based globus toolkit (hereinafter referred to as ‘GT4’). When a resource use specification for task processing arrives at the GT4, an electrocardiographic analysis algorithm for high-performance electrocardiographic analysis is executed. Here, whilst the analysis performed by the electrocardiographic analysis block 1023 in FIG. 2 is a fast-track analysis on the human body, the analysis performed by the task dispatcher 1063-4 is a relatively high-performance analysis such as multi-channel and/or long-time electrocardiographic analysis. That is, for the realization of the high-performance diagnosis services, the electrocardiographic analysis module 1063 in the medical service server 106 produces an electrocardiographic analysis result through high-performance electrocardiographic analysis in a series of processes described above. The produced electrocardiographic analysis result is transferred to an agreement analyzer 1064-5 (FIG. 6) in the virtual heart simulation module 1064 and to a diagnosis result correction block 1065-1 (FIG. 7) in the cardiovascular disorder diagnosis module 1065.

Referring back to FIG. 3, the virtual heart simulation module 1064 performs a virtual heart simulation on the basis of information on user requested task such as the virtual heart simulation parameters and the like, a user service level, information on computing resources allocated to the task, and scheduling information, and derives a pseudo electrocardiogram and magnetocardiogram. The virtual heart simulation module 1064 performs an analysis of agreement between the electrocardiographic analysis result and real magnetocardiographic treatment data, and the pseudo electrocardiogram and magnetocardiogram, determines the disease state of cardiovascular disorders of the human body in accordance with the degree of agreement, and sends the disease state information to the cardiovascular disorder diagnosis module 1065. These functions are described further in connection with FIG. 6.

FIG. 6 is a detail block diagram illustrating the virtual heart simulation module 1064 in FIG. 3.

Referring to FIG. 6, the virtual heart simulation module 1064 includes a virtual heart simulator 1064-1, resource selector 1064-2, task allocator 1064-3, task dispatcher 1064-4, agreement analyzer 1064-5, and virtual heart disease diagnostics 1064-6.

The virtual heart simulator 1064-1 receives virtual heart simulation parameters from the Web service block 1061, and sends the received virtual heart simulation parameters to the resource selector 1064-2. The simulation parameters is used to build a pathological model for cardiovascular disorders (for example, ischemia, PVC, LBBB, tachycardia, and bradycardia), and may include a cardiac cycle (msec), ischemic region, region of purkinje fibers (or the number of a purkinje fiber having self stimuli) at which PVC occurs, calcium concentration at the calcium channel, potassium concentration, slow potassium concentration, and sodium concentration.

The simulation parameters may be diagnostic parameters arbitrarily assigned by the user requesting a high-performance cardiovascular disorder diagnosis service, or partially modified versions of diagnostic parameters obtained by actual diagnosis of the human body. These assigned and modified diagnostic parameters are provided to the virtual heart simulator 1064-1) in the medical service server 106 via the network 104 from a corresponding client.

The resource selector 1064-2 selects the computing resources to be used for the virtual heart simulation on the basis of the user requested task information from the virtual heart simulator 1064-1 and the optimum available resource information from the MDP-based quorum generation module 1062-21 in FIG. 4. Information regarding the selected task and resource is transferred to the task allocator 1064-3.

The task allocator 1064-3 selects resources to be allocated on the basis of the scheduling information from the task scheduler 1062-32 in FIG. 4 with respect to task information and resource selection information from the resource selector 1064-2. The resource-to-task assignment information is transferred to the task dispatcher 1064-4.

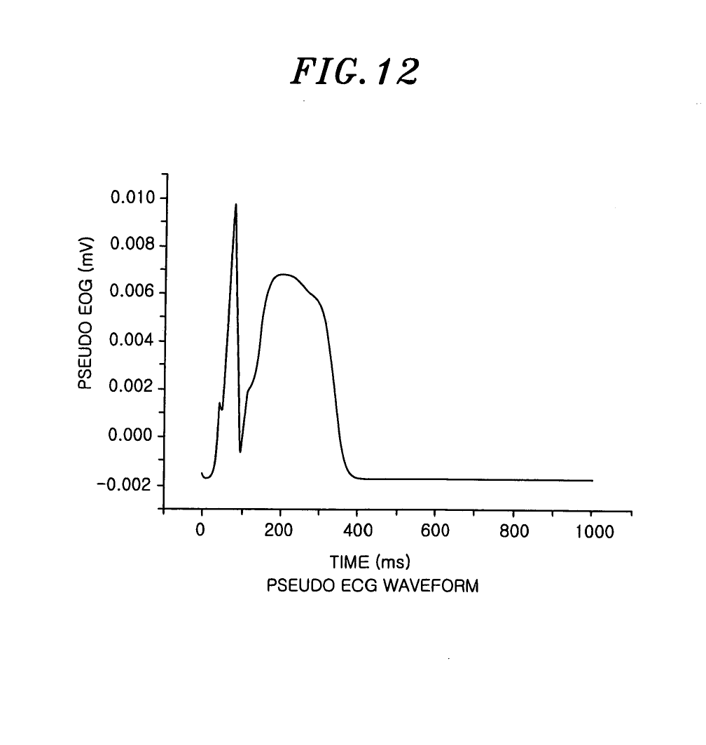

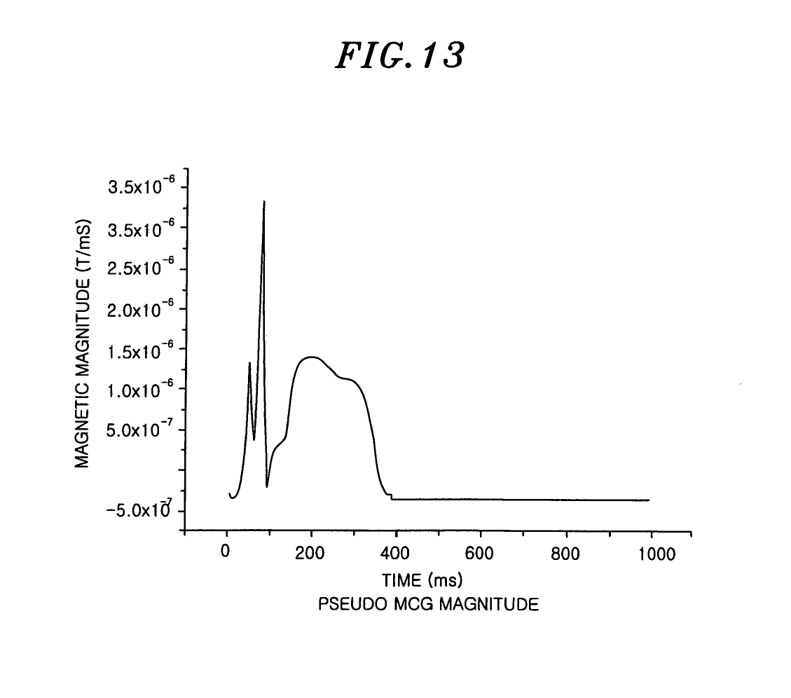

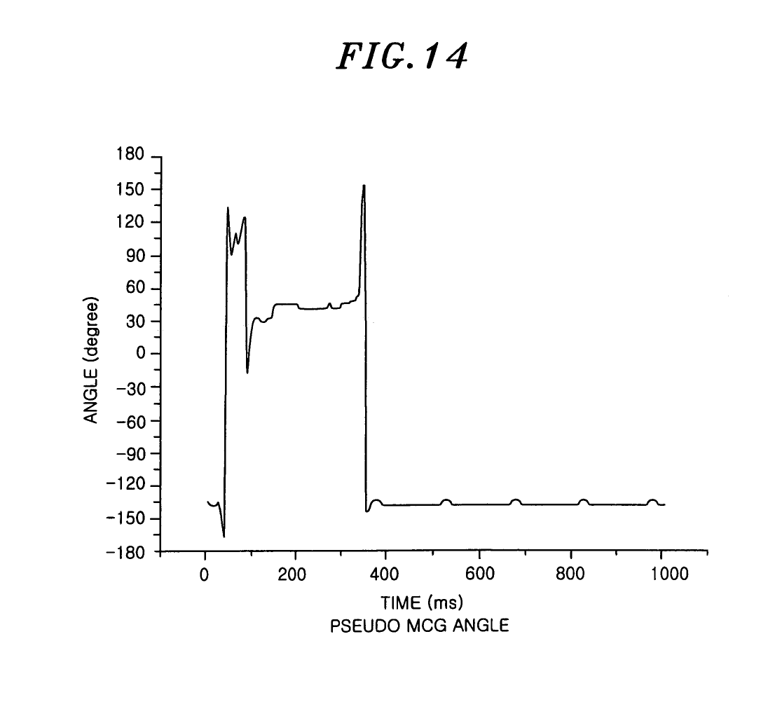

The task dispatcher 1064-4 performs a virtual heart simulation in a distributed manner using, for example, a Grid middleware-based Globus toolkit (GT4). When a resource use specification for task processing arrives at the GT4, a high-performance virtual heart simulation is performed by way of the execution of an electrocardiogram and magnetocardiogram derivation algorithm to thereby derive a pseudo electrocardiogram and magnetocardiogram. The pseudo electrocardiogram and magnetocardiogram information (waveform information) derived by the virtual heart simulation is transferred to the agreement analyzer 1064-5. For example, information including a pseudo electrocardiographic waveform shown in FIG. 12, a pseudo magnetocardiographic waveform shown in FIG. 13, and a pseudo magnetocardiographic angle waveform shown in FIG. 14 is created through the virtual heart simulation, and transferred to the agreement analyzer 1064-5.

The agreement analyzer 1064-5 performs an analysis of agreement between the real magnetocardiographic treatment data (the real magnetocardiographic waveform information) from the Web service block 1061 in FIG. 3, the electrocardiographic analysis result from the task dispatcher 1063-4 in FIG. 5, and the pseudo electrocardiogram and magnetocardiogram from the task dispatcher 1064-4, through signal processing. The agreement analyzer 1064-5 sends the agreement analysis result to the virtual heart disease diagnostics 1064-6.

The virtual heart disease diagnostics 1064-6 determines the disease state of cardiovascular disorders of the human body in accordance with an agreement analysis result from the agreement analyzer 1064-5. That is, the disease state is determined by the initial parameters to the virtual heart simulation in accordance with the degree of agreement between the real electrocardiogram and magnetocardiogram and the pseudo electrocardiogram and magnetocardiogram. The determined initial disease state information on cardiovascular disorders is transferred to Figto a diagnosis result corrector block 1065-1 (FIG. 7) in the cardiovascular disorder diagnosis module 1065 in FIG. 4Fig.

Referring back to FIG. 3, the cardiovascular disorder diagnosis module 1065 performs a definitive cardiovascular disorder diagnosis on the human body on the basis of the real magnetocardiographic treatment data, the electrocardiographic analysis result from the electrocardiographic analysis module, the disease state from the virtual heart simulation module and a diagnosis criteria from a diagnosis reference table, and provides the definitive diagnosis result through the network to the client requesting the high-performance diagnosis service. These functions are described further in connection with FIG. 7.

FIG. 7 is a detail block diagram illustrating the cardiovascular disorder diagnosis module 1065 in FIG. 3.

Referring to FIG. 7, the cardiovascular disorder diagnosis module 1065 includes a diagnosis result corrector 1065-1, definitive diagnostics 1065-2, and diagnosis reference table 1065-3.

The diagnosis result corrector 1065-1 performs a selective corrective operation on the basis of relations among the real magnetocardiographic treatment data from the Web service block 1061 in FIG. 3, the electrocardiographic analysis result from the task dispatcher 1063-4 in FIG. 5, and the disease state information from the virtual heart disease diagnostics 1064-6 in FIG. 6. For example, if relations among the real magnetocardiogram, the electrocardiographic analysis result, and the disease state represent a noticeable disparity or if the diagnosis date is too old, the diagnosis result corrector 1065-1 may request the corresponding client to perform another diagnosis on the human body, or reflect this condition in the definitive diagnosis of cardiovascular disorders.

The definitive diagnostics 1065-2 performs a definitive cardiovascular disorder diagnosis on the human body on the basis of the real magnetocardiogram, the electrocardiographic analysis result and the disease state information or corrected versions of these from the diagnosis result corrector 1065-1, and the diagnosis criteria from the diagnosis reference table 1065-3. For example, when the electrocardiographic analysis shows a heart rate variability (HRV) of higher than or equal to the reference value and not too serious ST-T segment changes, and when the magnetocardiographic analysis shows a subtle tendency of an ischemic disease (such as maximum current moment, maximum current and the like), the definitive diagnostics 1065-2 finds probable regions having ischemic symptoms, checks the severity of ischemia, and issues a definitive diagnosis using the diagnosis reference table 1065-3.

In addition, the definitive diagnostics 1065-2 checks the abnormality of diagnostic results (for example, ST-wave, P-wave, and U-wave) obtained from the electrocardiographic analysis of cardiovascular disorders, and also checks the abnormality of diagnostic results (for example, current moment dynamics, current angle maximum, and current angle minimum) obtained from the magnetocardiographic analysis.

Therefore, the diagnosis reference table 1065-3 stores various diagnosis criteria in a tabular form for cardiovascular disorder diagnoses. The definitive diagnostics 1065-2 collects definitive diagnosis result data on cardiovascular disorders of the human body, and sends the collected definitive diagnosis result data through the Web service block 1061 and network 104 to the client requesting a high-performance diagnosis service. Diagnostic catalog information regarding the definitive diagnosis result data on cardiovascular disorders (for example, treatment hospital name, and patient name, sex, etc) is transferred through the Web service block 1061 to the distributed-data processing module 1066, which then stores the diagnostic catalog information in the data catalog storage block 1067.

Accordingly, the corresponding user can readily receive the result of a high-performance diagnosis on cardiovascular disorders of a human body being a treatment object through a series of steps described above.

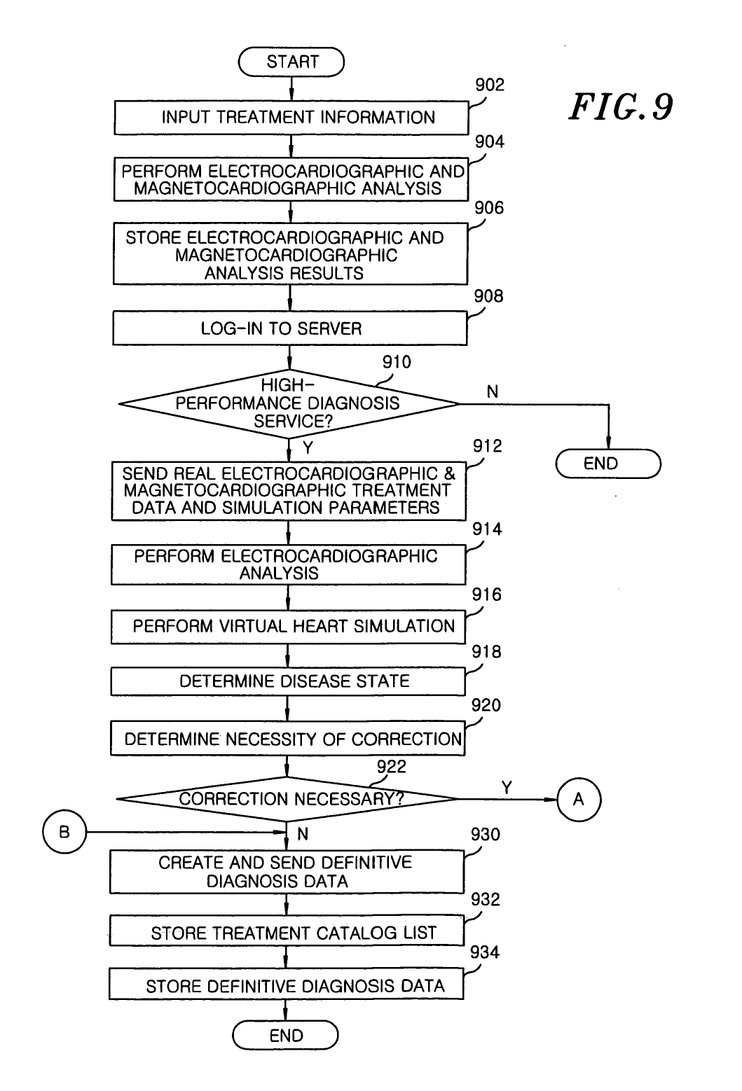

Referring back to FIG. 3, the distributed-data processing module 1066 provides an integrated data management service for definitive diagnosis data on cardiovascular disorders that is stored in data repositories distributed at multiple sites on the basis of location and type information on data repositories from the data catalog storage block 1067. This function is described further in connection with FIG. 8.

The data catalog storage block 1067 corresponds to a catalog database for storing diagnosis data storage information. The data catalog storage block 1067 stores location information (e.g., IP addresses) and type information (e.g., MySql, MsSql and the like) of data repositories located at different sites, and diagnosis catalog information. The type information is used to select a suitable driver for a data repository, and the diagnosis catalog information denotes a diagnosis list having hospital names, and patient names and sexes of human bodies. Whenever the state of definitive diagnosis data in each data repository (i.e., the diagnosis data storage block of a client) is changed in part and addition, the diagnosis data storage information stored in the data catalog storage block 1067 is updated accordingly using the changed information from the distributed-data processing module 1066.

FIG. 8 is a block diagram illustrating the distributed-data processing module 1066 in FIG. 3.

As shown in FIG. 8, the distributed-data processing module 1066 includes a data request analyzer 1066-1, data access controller 1066-2, and distributed-data request handler 1066-3.

The data request analyzer 1066-1 analyzes an access request for diagnosis data from the Web service block 1061 in FIG. 3, and sends the access request to the data access controller 1066-2. Upon access request reception from a user, the data access controller 1066-2 receives information necessary for a user access control (e.g., service class-based access control) from the SLA information storage 1062-12 and user information storage 1062-13 in FIG. 4, and verifies the adequacy of access rights of the requesting user on the basis of the received information.

If it is decided that the requesting user has adequate access rights, the data request analyzer 1066-1 receives the location and the type information of a data repository of a client having the requested diagnosis data, analyzes the received location and type information, and then sends a data use request to a corresponding distributed-data request handler 1066-3.

Although only one distributed-data request handler 1066-3 is illustrated in FIG. 8 for the purpose of illustration, the medical service server 106 may includes a plurality of distributed-data request handling blocks 1066-3. Substantially, the data request analyzer 1066-1 may simultaneously send the data use request to one or more distributed-data request handling blocks. The data use request means retrieval of desired diagnosis data from a data repository, modification to existing diagnosis data in a data repository, or addition of new diagnosis data to a data repository.

The distributed-data request handler 1066-3 creates a data use request command, and sends the data use request command through the Web service block 1061 and the network 104 to a data repository of a corresponding client in the client group 102. When the requested diagnosis data is received from the corresponding client, the distributed-data request handler 1066-3 forwards the received diagnosis data through the Web service block 1061 and the network 104 to the requesting client.

The user of a client can input the name of a human body after logging-in, send the name to the medical service server 106, and receive definitive diagnosis data on cardiovascular disorders of the human body, which is delivered from a client having the desired definitive diagnosis data of the human body via the medical service server 106. The client can also select the name of the human body from a treatment catalog list presented by the medical service server 106, and receive the definitive diagnosis data on cardiovascular disorders of the selected human body. The user is able to receive definitive diagnosis data from a remote data repository and may be limited to, for example, a medical specialist having an adequate data access right under user access control.

In the description of the present embodiment, the data catalog storage block is located at the medical service server. However, the present invention is not limited thereto. That is, the data catalog storage block may also be located at a remote server or computer external to the medical service server.

According to the present invention, the cardiovascular disorder diagnosis system having the above-described configuration can provide the user with an efficient integrated management service for various cardiovascular disorder diagnosis data distributed among multiple data repositories through a series of processes described previously.

Further, in the description of the cardiovascular disorder diagnosis system, it has been described and shown that the client sends the real electrocardiographic and magnetocardiographic treatment data and the virtual heart simulation parameters of the human body to the medical service server and receive a high-performance cardiovascular disorder diagnosis service. However, the present invention is not necessarily limited thereto. The client can also receive the high-performance cardiovascular disorder diagnosis service by sending only the real electrocardiographic and magnetocardiographic treatment data of the human body to the medical service server. A differentiated service like this may be based on a corresponding service level and service class. In this case, the medical service server creates an electrocardiographic analysis result using the received real electrocardiographic treatment data, and performs a definitive diagnosis on cardiovascular disorders of the human body on the basis of the electrocardiographic analysis result and the real magnetocardiographic treatment data. To do it, the diagnosis reference table in the medical service server is required to store corresponding diagnosis standard information (i.e., enabling definitive cardiovascular disorder diagnosis based on the electrocardiographic analysis result and the real magnetocardiographic treatment data only Figwithout the virtual heart simulation module in the medical service server of FIG. 3.

Hereinafter, procedures for providing a client with a high-performance diagnosis service using the cardiovascular disorder diagnosis system will be described.

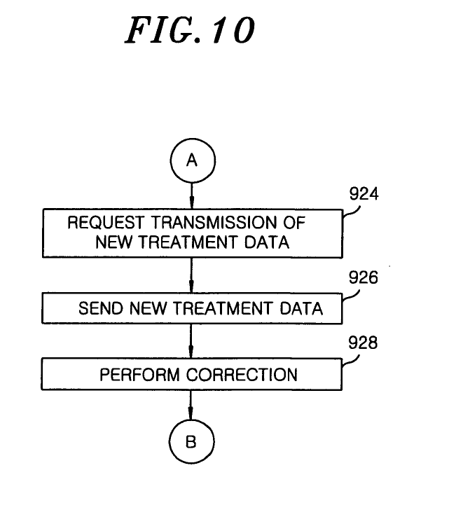

FIGS. 9 and 10 are flow charts illustrating a procedure of providing a high-performance diagnosis service for cardiovascular disorders to clients in accordance with another embodiment of the present invention.

In FIG. 9, first of all, when treatment information of a human body being a treatment object that is obtained through a medical instrument for a cardiovascular disorder diagnosis is provided to a client, the control block 1022 of the client sends the treatment information to the electrocardiographic analysis block 1023 and the magnetocardiographic analysis block 1025 (step 902).

The electrocardiographic analysis block 1023 analyzes electrocardiographic signals using an electrocardiographic analysis algorithm, and the magnetocardiographic analysis block 1025 analyzes magnetocardiographic signals using a magnetocardiographic analysis algorithm (step 904). The electrocardiographic analysis block 1023 stores the electrocardiographic analysis result in the electrocardiographic information storage block 1024 as real electrocardiographic treatment data of the human body, and the magnetocardiographic analysis block 1025 stores the magnetocardiographic analysis result in the magnetocardiographic information storage block 1026 as real magnetocardiographic treatment data of the human body (step 906).

The user (a doctor having valid diagnosis service usage rights) logs in to the medical service server 106 through the network 104 (step 908). If the user requests a high-performance cardiovascular disorder diagnosis service by inputting virtual heart simulation parameters (step 910), the control block 1022 retrieves the real electrocardiographic treatment data and the real magnetocardiographic treatment data respectively from the electrocardiographic information storage block 1024 and magnetocardiographic information storage block 1026, and sends the real electrocardiographic and magnetocardiographic treatment data and virtual heart simulation parameters along with a diagnosis service request through the Web service block 1027 and network 104 to the Web service block 1061 (FIG. 3) in the medical service server 106 (step 912).

The Web service block 1061 forwards the real electrocardiographic treatment data to the electrocardiographic analysis module 1063, and also forwards the real magnetocardiographic treatment data to the virtual heart simulation module 1064 and cardiovascular disorder diagnosis module 1065.

The electrocardiographic analysis module 1063 analyzes the real electrocardiographic treatment data through Grid-based electrocardiographic analysis on the basis of user-requested task information from the Web service block 1061 and information regarding a user service level, an available computing resource, and a task schedule from the information storage/management module 1062, and sends the electrocardiographic analysis result to the virtual heart simulation module 1064 and cardiovascular disorder diagnosis module 1065 (step 914).

More specifically, in step 914, for the electrocardiographic analysis, resources to be used are selected on the basis of the user-requested task information and optimum available resource information (i.e., that is created from resource state information, resource quality information and task history information) from the information storage/management module 1062. Resources to be allocated are selected on the basis of task information, resource selection information, and scheduling information from the information storage/management module 1062. Tasks related to the Grid middleware-based electrocardiographic analysis of the real electrocardiographic treatment data are processed in a distributed manner using the resource-to-task assignment information, thereby creating an electrocardiographic analysis result.

Thereafter, the virtual heart simulation module 1064 performs a virtual heart simulation on the basis of the user-requested task information, the user service level information, the computing resource-to-task assignment information and the scheduling information from the Web service block 1061, to thereby derives the pseudo electrocardiogram and magnetocardiogram (step 916). The virtual heart simulation module 1064 then determines the disease state of cardiovascular disorders in the human body through an analysis of agreement between the electrocardiographic analysis result, real magnetocardiographic treatment data, and pseudo electrocardiogram and magnetocardiogram, and sends the disease state information to the cardiovascular disorder diagnosis module 1065 (step 918).

More specifically, in step 916, for the virtual heart simulation, resources to be used are selected on the basis of the user requested task information and the optimum available resource information from the information storage/management module 1062. In addition, resources to be allocated are selected on the basis of the task information, the resource selection information, and the scheduling information from the information storage/management module 1062. Tasks related to the Grid middleware-based virtual heart simulation are processed in a distributed manner using the resource-to-task assignment information, thereby deriving a pseudo electrocardiogram and magnetocardiogram as in FIGS. 12 and 13. Here, the simulation parameters may be diagnostic parameters assigned by the user (doctor) requesting a high-performance cardiovascular disorder diagnosis service, or partially modified versions of diagnostic parameters obtained by an actual diagnosis of a human body being a treatment object.

Subsequently, in step 918, an analysis of agreement is performed through signal processing between real magnetocardiographic treatment data of the human body (real magnetocardiographic waveform information) from the Web service block 1061, the electrocardiographic analysis result (real magnetocardiographic waveform analysis information) from the electrocardiographic analysis module 1063, and the pseudo electrocardiogram and magnetocardiogram (waveform information). The disease state of the human body is determined in accordance with the agreement analysis result.

In the description of the present embodiment, the electrocardiographic analysis is performed before the virtual heart simulation. However, the present invention is not necessarily limited thereto. It is noted that the electrocardiographic analysis and virtual heart simulation are concurrently performed in practice.

Thereafter, the cardiovascular disorder diagnosis module 1065 checks whether or not there needs a correction to the real treatment data (step 920). For example, if the relations among the real magnetocardiogram, the electrocardiographic analysis result, and the disease state represent a noticeable disparity or if the diagnosis date is too old, the cardiovascular disorder diagnosis module 1065 can determine the necessity of correction.

If the correction is necessary in step 922, a control process goes through a tab “A” to step 924, where the cardiovascular disorder diagnosis module 1065 sends a request message for new real treatment data to the corresponding client. The requested treatment data may be real electrocardiographic treatment data, real magnetocardiographic treatment data, and a combination of these.

In response thereto, the corresponding client creates the requested treatment data, and sends the treatment data to the medical service server 106 (step 926), and then selective corrections are made (step 928). In subsequent steps 926 and 928, in the case when the requested treatment data is the real magnetocardiographic treatment data, the new treatment data is sent again to the virtual heart simulation module 1064 and the cardiovascular disorder diagnosis module 1065; the virtual heart simulation is performed once again; and the definitive cardiovascular disorder diagnosis is performed accordingly. In the case where the requested treatment data is the real electrocardiographic treatment data, the new treatment data is sent again to the electrocardiographic analysis module 1063; a new electrocardiographic analysis is performed; and a definitive cardiovascular disorder diagnosis is performed accordingly. In the case where the requested treatment data is the real magnetocardiographic and electrocardiographic treatment data, the new treatment data is sent to the electrocardiographic analysis module 1063, the virtual heart simulation module 1064 and the cardiovascular disorder diagnosis module 1065; and the electrocardiographic analysis, the virtual heart simulation, and the definitive cardiovascular disorder diagnosis are performed once again.

In step 922, if none of the correction is needed, a control process advances through a tab “B” to step 930, where the cardiovascular disorder diagnosis module 1065 performs the definitive cardiovascular disorder diagnosis of the human body on the basis of the real magnetocardiographic treatment data, the electrocardiographic analysis result, the disease state information (or corrected versions of these) and the diagnosis criteria from the diagnosis reference table, and transmits the definitive cardiovascular disorder diagnosis result through the network 104 to the corresponding client.

Further, the cardiovascular disorder diagnosis module 1065 creates diagnostic catalog data containing the location and type of a repository, treatment hospital name, and patient name and sex, and transmits the diagnostic catalog data to the distributed-data processing module 1066, which then stores the diagnostic catalog data in the data catalog storage block 1067 (step 932). The diagnostic catalog data is used as integrated data management information that enables a client having adequate usage rights to use various definitive cardiovascular disorder diagnosis data obtained through high-performance analyses that are distributed among data repositories of the other clients).

The corresponding client requesting the high-performance diagnosis service stores the high-performance definitive diagnosis data on cardiovascular disorders, received through the network 104 from the medical service server 106, in the diagnosis data storage block 1028 (step 934). Therefore, the user of the corresponding client can readily receive the high-performance definitive diagnosis result for the human body being a treatment object, and view the diagnosis result displayed on a display panel (not shown).

Accordingly, the diagnosis service method for cardiovascular disorders of the present invention enables a user to rapidly receive a high-performance cardiovascular disorder diagnosis service for the human body through a series of processes described above.

In the diagnosis service method for cardiovascular disorders, it has been described and shown that a client sends real electrocardiographic and magnetocardiographic treatment data and virtual heart simulation parameters of the human body through the network to the medical service server in order to receive a high-performance cardiovascular disorder diagnosis service. However, the present invention is not necessarily limited thereto. Similarly to the case of the diagnosis service providing system, the client can also receive a high-performance cardiovascular disorder diagnosis service by sending only real electrocardiographic and magnetocardiographic treatment data of a human body to the medical service server. A differentiated service like this may be based on a corresponding service level and service class.

Next, a procedure is described for providing a client with an integrated data management service for high-performance diagnosis data distributed among multiple data repositories.

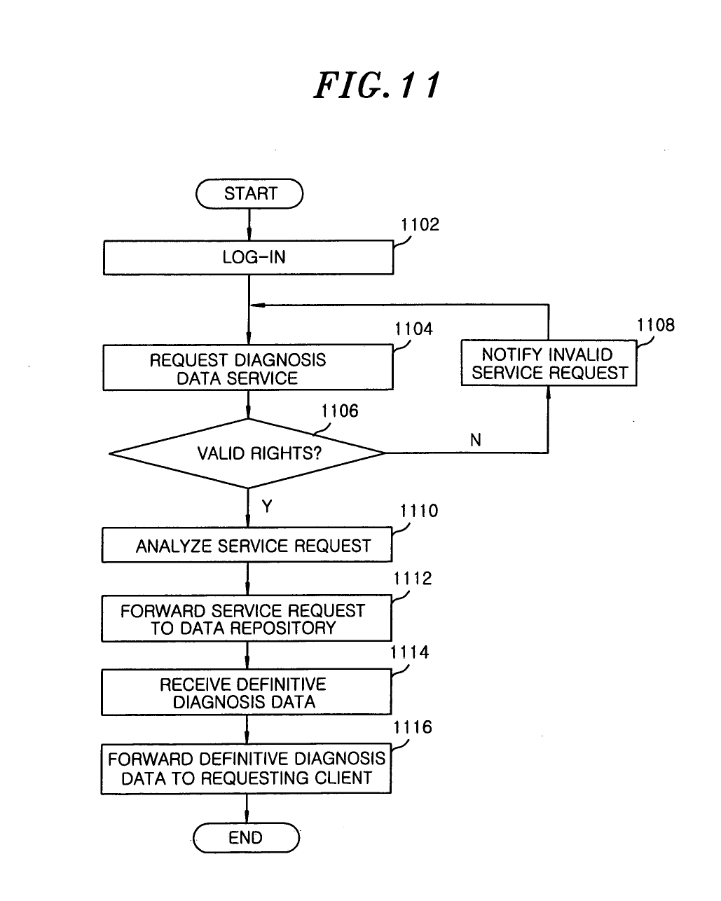

FIG. 11 is a flow chart illustrating a procedure of providing a client with an integrated management service for cardiovascular disorder diagnosis data that is stored in a plurality of distributed data repositories.

As shown in FIG. 11, if the user of a client connects through the network 104 to the medical service server 106 and logs in thereto, the medical service server 106 provides the client with a main menu screen containing a service request menu item for definitive diagnosis result data (step 1102).

The user of the client requests desired diagnosis data by selecting the service request menu item in the main menu (step 1104). The distributed-data processing module 1066 checks whether or not the user has a valid usage right for the service request, through authentication using the information storage/management module 1062 (step 1106).

If it is checked that the user does not have a valid usage right, the distributed-data processing module 1066 sends a notification message indicating an invalid usage right to the client (step 1108).

However, if it is checked that the user has the valid usage right, the distributed-data processing module 1066 analyzes the diagnosis data request from the client with reference to the data catalog storage block 1067, and extracts the location and type information of a data repository of a client having the desired diagnosis data (step 1110).

In step 1110, the user of the client can select desired diagnosis data by referring to the diagnosis catalog list or by directly inputting the name of a human body being a treatment object. For catalog list use, the distributed-data processing module 1066 creates a diagnosis catalog list using information from the data catalog storage block 1067, and sends the diagnosis catalog list to the client. Then, the user of the client selects one or more items in the diagnosis catalog list.

Thereafter, the distributed-data processing module 1066 forwards the diagnosis data request to the client having the extracted location and type information (step 1112). The requested client retrieves the requested diagnosis data from the diagnosis data storage block, and sends the retrieved diagnosis data to the distributed-data processing module 1066 (step 1114).

Subsequently, the distributed-data processing module 1066 sends the diagnosis data from the requested client to the requesting client, and stores a tag including the identifier of the used data item, used date and user in the data catalog storage block 1067 (step 1116). Whenever the diagnosis data is utilized by any clients, a tag is created and saved in the data catalog storage block 1067 to manage the usage history of the diagnosis data.

Accordingly, the diagnosis service method for cardiovascular disorders of the present invention provides a user with an efficient integrated management service for various cardiovascular disorder diagnosis data distributed among multiple data repositories through a series of steps described above.

Next, an example is described of applying the diagnosis service method of the present invention.

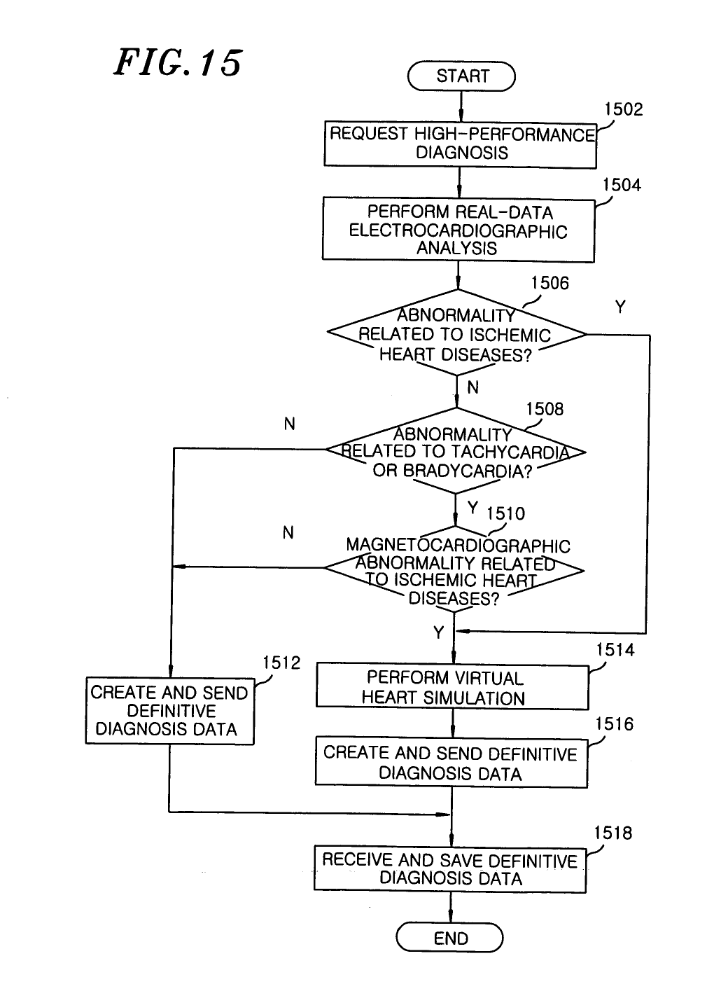

FIG. 15 is a flow chart illustrating a procedure of providing a diagnosis service for tachycardia, bradycardia and ischemic heart diseases through selective performance of an electrocardiographic analysis and virtual heart simulation.

As shown in FIG. 15, the user of a client having a valid service usage right connects through the network 104 to the medical service server 106 and logs in thereto, and sends real electrocardiographic and magnetocardiographic treatment data and virtual heart simulation parameters of a human body being a treatment object to the medical service server 106 as part of a high-performance diagnosis request for cardiovascular disorders (step 1502).

The electrocardiographic analysis module 1063 performs an analysis on the real electrocardiographic treatment data in a distributed manner (Grid middleware-based distributed processing) with reference to various information from the information storage/management module 1062, generates an electrocardiographic analysis result, and sends the electrocardiographic analysis result to the virtual heart simulation module 1064 and cardiovascular disorder diagnosis module 1065 (step 1504).

After that, the cardiovascular disorder diagnosis module 1065 checks whether or not there is the presence of abnormalities associated with ischemic heart diseases on the basis of the electrocardiographic analysis result from the electrocardiographic analysis module 1063 and a diagnosis criteria from the diagnosis reference table (step 1506).

If the abnormalities associated with ischemic heart diseases are not detected, the cardiovascular disorder diagnosis module 1065 checks whether or not there is the presence of abnormalities associated with tachycardia or bradycardia on the basis of diagnosis criteria from the diagnosis reference table (step 1508). If the abnormalities associated with tachycardia or bradycardia are not detected, the cardiovascular disorder diagnosis module 1065 creates definitive diagnosis data indicating a normal state of the human body, and sends the definitive diagnosis data to the requesting client (step 1512). As a result, the user of the client is notified of absence of cardiovascular disorders in the human body using the definitive diagnosis data (step 1518).

In this regard, before or after transmission of the definitive diagnosis data, the cardiovascular disorder diagnosis module 1065 may create diagnosis catalog information (including, for example, the location and type of a data repository, treatment hospital name, and name and sex of the human body) corresponding to the definitive diagnosis data, and save the diagnosis catalog information at its own data catalog storage block. The requesting client may also save the definitive diagnosis data at its own diagnosis data storage block.

If, however, abnormalities associated with tachycardia or bradycardia are detected at step 1508, the cardiovascular disorder diagnosis module 1065 checks whether or not there is the presence of abnormalities associated with ischemic heart diseases on the basis of the real magnetocardiographic treatment data and diagnosis criteria from the diagnosis reference table (step 1510).

If it is checked that abnormalities associated with ischemic heart diseases are not detected, the cardiovascular disorder diagnosis module 1065 creates definitive diagnosis data containing an indication of tachycardia or bradycardia in the human body, and sends the definitive diagnosis data to the requesting client (step 1512). As a result, the user of the client is notified of an indication of tachycardia or bradycardia in the human body (step 1518).

In this regard, before or after transmission of the definitive diagnosis data, the cardiovascular disorder diagnosis module 1065 may create diagnosis catalog information (including, for example, the location and type of a data repository, treatment hospital name, and name and sex of the human body) corresponding to the definitive diagnosis data, and may save the diagnosis catalog information at its own data catalog storage block. The requesting client may also save the definitive diagnosis data at its own diagnosis data storage block.

If it is checked that abnormalities associated with ischemic heart diseases are detected by magnetocardiography at step 1510, the virtual heart simulation module 1064 performs, under the command of the cardiovascular disorder diagnosis module 1065, a virtual heart simulation using the input parameters and various information from the information storage/management module 1062 in a distributed manner to derive a pseudo electrocardiogram and magnetocardiogram; determines the disease state of cardiovascular disorders of the human body through an analysis of agreement between the real magnetocardiographic treatment data, electrocardiographic analysis result, and pseudo electrocardiogram and magnetocardiogram; and sends the disease state information to the cardiovascular disorder diagnosis module 1065 (step 1514).

Thereafter, the cardiovascular disorder diagnosis module 1065 creates high-performance definitive diagnosis data through comparison between the real magnetocardiographic treatment data, electrocardiographic analysis result, pseudo electrocardiogram and magnetocardiogram, and diagnosis criteria from the diagnosis reference table, and sends the definitive diagnosis data to the requesting client (step 1516). The created definitive diagnosis data is saved as diagnosis catalog data at the data catalog storage block of the medical service server 106.

As a result, the user of the client is notified of the state of cardiovascular disorders in the human body (step 1518). The definitive diagnosis data is then stored at its own diagnosis data storage block for integrated management for later use by itself or other clients.

As described above, according to the present embodiment, the user of a client can receive a high-performance diagnosis service for tachycardia, bradycardia and ischemic heart diseases by sending real electrocardiographic and magnetocardiographic treatment data of a human body being a treatment object to the medical service server.

While the invention has been shown and described with respect to the embodiments, it will be understood by those skilled in the art that various changes and modifications may be made without departing from the spirit and scope of the invention as defined in the following claims.

What is claimed is:

1. A diagnosis system for providing cardiovascular disorder diagnosis services through a network, comprising:
a client group having one or more clients, each of which transmits real electrocardiographic treatment data and magnetocardiographic treatment data of a human body being a treatment object along with a cardiovascular disorder diagnosis request through the network, receives definitive diagnosis data as a reply to the cardiovascular disorder diagnosis request through the network; and
a medical service server for analyzing the real electrocardiographic treatment data received through the network from the client in accordance with a task schedule utilizing available resource information, determining a disease state of the human body on the basis of the electrocardiographic analysis result, the real magnetocardiographic treatment data, and pseudo electrocardiogram and magnetocardiogram obtained through a virtual heart simulation, creating definitive diagnosis data on cardiovascular disorders of the human body on the basis of the real magnetocardiographic treatment data, the electrocardiographic analysis result and the determined disease state, and transmitting the created definitive diagnosis data through the network to the client.

2. The diagnosis system of claim 1, wherein each of the clients comprises:
an electrocardiographic analysis block for analyzing an electrocardiographic signal in the treatment data to generate the real electrocardiographic treatment data;
a magnetocardiographic analysis block for analyzing a magnetocardiographic signal in the treatment data to generate the real magnetocardiographic treatment data;
a control block for transmitting the retrieved real electrocardiographic treatment data and the magnetocardiographic treatment data and virtual heart simulation parameters provided thereto along with the cardiovascular disorder diagnosis request to the medical service server, and receiving the definitive diagnosis data to be delivered to the client from the medical service server; and
a diagnosis data storage block for storing the definitive diagnosis data.

3. A method of providing cardiovascular disorder diagnosis services through a network, comprising:
requesting, by a client, a high-performance diagnosis on cardiovascular disorders by transmitting real electrocardiographic treatment data and magnetocardiographic treatment data of a human body being a treatment object and virtual heart simulation parameters through the network to a medical service server;
analyzing, by the medical service server, in response to the high-performance diagnosis request, the real electrocardiographic treatment data to generate an electrocardiographic analysis result;
performing, by the medical service server, a virtual heart simulation using the simulation parameters to generate a pseudo electrocardiogram and magnetocardiogram;
determining, by the medical service server, a disease state of the human body on the basis of the electrocardiographic analysis result, the magnetocardiographic treatment data, and the pseudo electrocardiogram and magnetocardiogram;
generating, by the medical service server, definitive diagnosis data for cardiovascular disorders through comparison between the real magnetocardiographic treatment data, the electrocardiographic analysis result, the disease state, and a diagnosis criteria; and
transmitting, by the medical service server, the definitive diagnosis data through the network to the client.

4. The method of claim 3, further comprising:
transmitting, by the other client, a data use request for desired diagnosis data on cardiovascular disorders to the medical service server;
extracting, by the medical service server, location and type information of the client having the desired diagnosis data stored therein through an analysis of the data use request with reference to a data catalog storage;
forwarding, by the medical service server, the data use request to the client on the basis of the extracted location and type information; and
receiving the diagnosis data related to the data use request from the client, and forwarding the received diagnosis data to the other client.

5. The method of claim 4, wherein the step of transmitting a data use request comprises:
connecting, by the other client, to the medical service server, and sending a request for a diagnosis catalog list having at least one diagnosis catalog;
sending, by the medical service server, the diagnosis catalog list retrieved from the data catalog storage to the other client; and
selecting, by the other client, a diagnosis catalog of the received diagnosis catalog list.

6. The method of claim 4, further comprising: writing tag information corresponding to a usage history of the received diagnosis data to the data catalog storage after forwarding of the received diagnosis data to the other client.

7. The method of claim 3, wherein the step of generating definitive diagnosis data comprises:
checking whether or not there needs a correction to the real treatment data through an analysis of relations among the real magnetocardiographic treatment data, the electrocardiographic analysis result, and the disease state;
generating, if there needs not the correction, the definitive diagnosis data through comparison among the real magnetocardiographic treatment data, the electrocardiographic analysis result, the disease state, and the diagnosis criteria;
sending, if there needs the correction, a request for new real treatment data of the human body to the client requesting a high-performance diagnosis; and
generating the definitive diagnosis data through comparison among the diagnosis data corrected in accordance with the new real treatment data from the client and the diagnosis criteria from the diagnosis reference table.

8. A method of providing cardiovascular disorder diagnosis services through a network, comprising:
requesting, by a client, a high-performance diagnosis on cardiovascular disorders by transmitting real electrocardiographic treatment data and magnetocardiographic treatment data of a human body being a treatment object and virtual heart simulation parameters through the network to a medical service server;
performing, by medical service server, in response to the high-performance diagnosis request, an analysis on the real electrocardiographic treatment data in a distributed manner to generate an electrocardiographic analysis result, and detecting whether or not there is an abnormality associated with ischemic heart diseases on the basis of the electrocardiographic analysis result and diagnosis criteria from a diagnosis reference table;
detecting, by medical service server, if the abnormality associated with the ischemic heart diseases is not detected, whether or not there is an abnormality associated with tachycardia or bradycardia on the basis of the diagnosis criteria from the diagnosis reference table;
creating, by medical service server, if the abnormality associated with tachycardia or bradycardia is not detected, definitive diagnosis data indicating a normal state of the human body, and sending the definitive diagnosis data through the network to the client;
detecting, by medical service server, if the abnormality associated with tachycardia or bradycardia is detected, whether or not there is an abnormality associated with ischemic heart diseases on the basis of the real magnetocardiographic treatment data and the diagnosis criteria from the diagnosis reference table;
creating, by medical service server, if the abnormality associated with ischemic heart diseases is not detected, definitive diagnosis data containing an indication of tachycardia or bradycardia in the human body, and sending the definitive diagnosis data through the network to the client;
deriving, by medical service server, if an abnormality associated with ischemic heart diseases is detected on the basis of the real electrocardiographic and/or magnetocardiographic treatment data, a pseudo electrocardiogram and magnetocardiogram through a distributed virtual heart simulation with the simulation parameters;
determining, by medical service server, a disease state of cardiovascular disorders of the human body on the basis of the electrocardiographic analysis result, the real magnetocardiographic treatment data, and the pseudo electrocardiogram and magnetocardiogram; and
creating, by medical service server, definitive diagnosis data through comparison among the real magnetocardiographic treatment data, the electrocardiographic analysis result, disease state and the diagnosis criteria, and sending the definitive diagnosis data through the network to the client.
